(19) United States
(12) Patent Application Publication
(10) Pub. No.: US 2011/0230496 A1
(43) Pub. Date: Sep. 22, 2011

(54) NOVEL PROCESS FOR PREPARING HIGHLY PURE LEVOCETIRIZINE AND SALTS THEREOF

(75) Inventors: Lior Zelikovitch, Mazkeret Batya (IL); Hila Shaked, Gadera (IL)

(73) Assignee: CHEMAGIS LTD., Bnei Brak (IL)

(21) Appl. No.: 12/671,664

(22) PCT Filed: Aug. 4, 2008
(86) PCT No.: PCT/IL08/01065

§ 371 Date: May 17, 2010

Related U.S. Application Data
(60) Provisional application No. 60/964,894, filed on Aug. 15, 2007.

(51) Int. Cl. A61K 31/495; C07D 295/14; A61P 37/08; A61P 11/06; A61P 11/02

(52) U.S. Cl. 514/255.04; 544/396

(57) ABSTRACT

A process for preparing pure levocetirizine and salts thereof, e.g., the levocetirizine dihydrochloride, and a pharmaceutical composition comprising levocetirizine dihydrochloride produced by the process are disclosed.

### CROSS-REFERENCE TO RELATED APPLICATIONS

This patent application claims the benefit of U.S. Provisional Patent Application No. 60/964,894, filed Aug. 15, 2007, which is incorporated by reference.

### FIELD OF THE INVENTION

The field of the invention relates to methods of purifying levocetirizine from a crude sample of levocetirizine via extraction.

### BACKGROUND OF THE INVENTION

Levocetirizine dihydrochloride is a third generation non-sedative antihistamine, developed from cetirizine, which is a second generation antihistamine. Levocetirizine is the active enantiomer of cetirizine, is more effective than cetirizine itself, and has fewer side effects.

Levocetirizine dihydrochloride blocks histamine receptors and is used for treating seasonal and perennial allergic rhinitis, chronic idiopathic urticaria, and for preventing and treating symptoms of allergic asthma. Levocetirizine dihydrochloride was first launched in 2001 by UCB, available as 5 mg tablets and marketed under the brand names XYZAL® in the United States, United Kingdom and France, and XUSAL® elsewhere in Europe.

The chemical name of levocetirizine, which is the R enantiomer of cetirizine, is (−)-2-[2-[4-[(4-chloropheyl)phenymethyl]-1-piperazinyl]-ethoxy]acetic acid, and it is represented by the following structural formula (I):

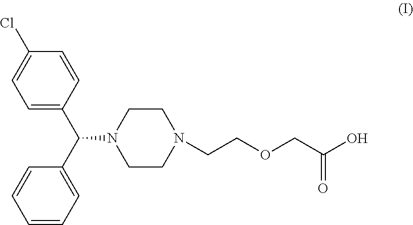

The preparation of cetirizine first was described in EP Patent No. 0 058 146, wherein the compound 2-[2-[4-[(4-chloropheyl)phenymethyl]-1-piperazinyl]-ethoxy]-acetamide (IV) was obtained by reacting 1-[(4-chlorophenyl)-phenylmethyl]-piperazine with a 2-haloethoxyacetic acid derivate, e.g., 2-(2-chloroethoxy)-acetamide (III), in xylene and sodium carbonate. Compound IV was hydrolyzed with potassium hydroxide to afford cetirizine potassium salt (V), which was acidified with HCl to obtain cetirizine dihydrochloride. The process is depicted in Scheme 1 below.

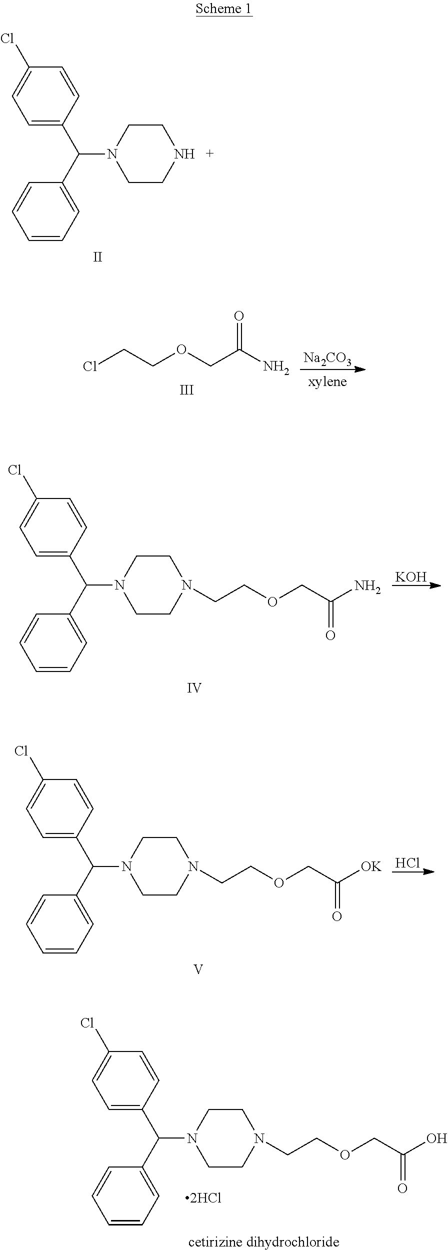

The preparation of cetirizine also is described in U.S. Pat. No. 6,100,400, comprising reacting Compound II with ethyl 2-chloroethoxyacetate DIA in a tertiary amine, such as triethylamine or triisopropylamine, to obtain ethyl 2-[2-[4-[(4-chloropheyl)-phenymethyl]-1-piperazinyl]-ethoxy]acetate IVA, which is further purified and hydrolyzed to afford cetirizine via cetirizine potassium salt (V), as depicted in Scheme 2 below.

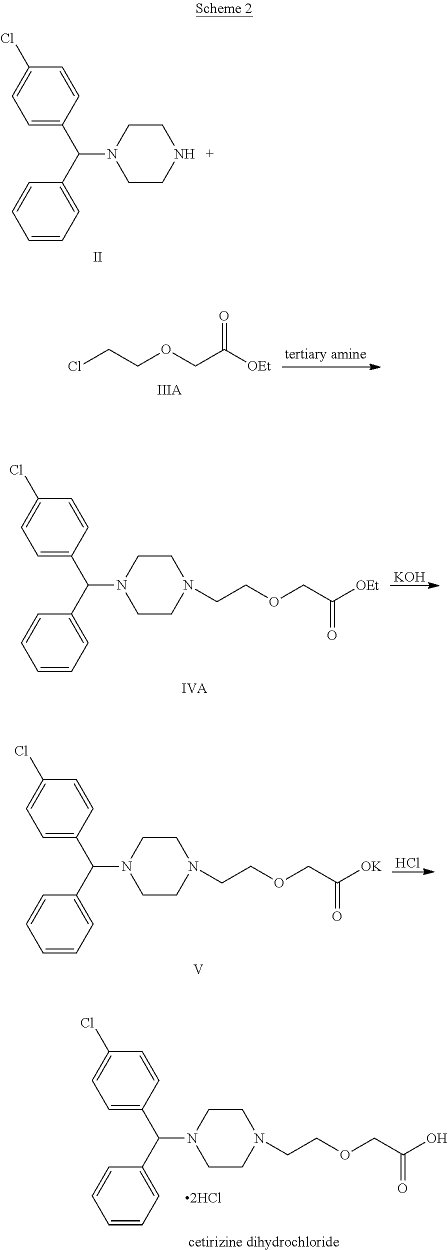

The preparation of levocetirizine is described in GB Patent No. 2,225,321, as depicted in Scheme 3 below. According to this synthetic route, the dextrorotatory 1-[(4-chlorophenyl)-phenylmethyl]-piperazine IIB is obtained by enantiomer resolution with tartaric acid in ethanol to obtain the intermediate 1-[(4-chlorophenyl)-phenylmethyl]-piperazine tartrate, which is purified by consecutive recrystallizations. The salt then is decomposed by treatment with sodium hydroxide (NaOH) in water, and the crude product is obtained by several extractions with dichloromethane and purified by consecutive re-crystallizations in hexane.

By heating the purified dextrorotatory 1-[(4-chlorophenyl)-phenylmethyl]-piperazine IIB with 2-chloroethoxyacetonitrile in the presence of sodium carbonate and potassium iodide in n-butanol, the dextrorotatory 2-[2-[4-[(4-chloropheyl)phenymethyl]-1-piperazinyl]-ethoxy]acetonitrile (Compound VI) is obtained. The dextrorotatory 2-[2-[4-[(4-chloropheyl)phenymethyl]-1-piperazinyl]-ethoxy]acetonitrile is heated in 37% hydrochloric acid, followed by addition of NaOH to afford free levocetirizine by extraction with several successive fractions of dichloromethane. Then, a solution of hydrochloric acid in acetone is added, and levocetirizine dihydrochloride is obtained. The disadvantages of this process are that it is very lengthy and provides the desired product in a relatively low enantiomeric purity of 95% by weight.

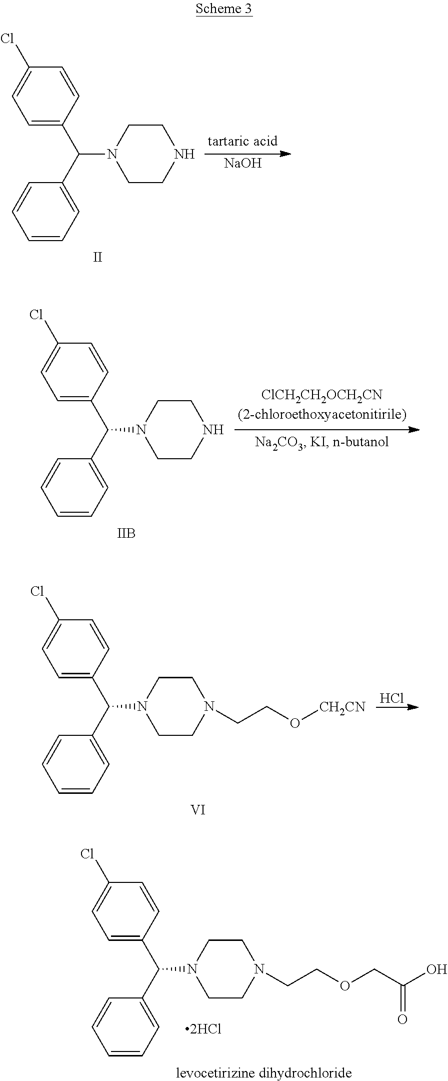

Due to the lengthy process and low optical purity of the known methods of synthesizing levocetrizine, there is a need in the art for an improved process for preparing highly chemically and highly enantiomerically pure levocetirizine that can be easily, conveniently, and inexpensively scaled-up for commercial production.

### SUMMARY OF THE INVENTION

It has been discovered that although precipitation of cetirizine potassium salt can be readily performed to enable its purification, the same procedure is not applicable for purifying the levocetirizine potassium salt. Therefore, an alternative approach is desirable for purifying the levocetirizine potassium salt in order to separate the levocetirizine potassium salt from impurities. Rather than precipitation, extraction has been discovered as a viable means of purifying levocetirizine from its impurities.

Thus, in one embodiment, the present invention provides a process for purifying levocetirizine or a salt thereof from a crude sample comprising levocetirizine, which comprises the steps of:
a) admixing a crude sample comprising levocetirizine potassium salt with a first organic solvent and water to form a first organic phase and a first aqueous phase;
b) separating the first aqueous phase from the first organic phase;
c) adding an acid and a second organic solvent to the first aqueous phase to form a second organic phase and a second aqueous phase;
d) separating the second organic phase from the second aqueous phase;
e) distilling the second organic solvent from the second organic phase to form a residue;
f) dissolving the residue of step (e) in a third organic solvent;
g) bubbling hydrogen chloride gas through the solution of step (f) to precipitate crystals of levocetirizine dihydrochloride; and
h) optionally isolating, washing, and drying the crystals. In some embodiments, the crystals are isolated by filtration.

In some embodiments, the crystals of levocetirizine dihydrochloride obtained using the methods as described herein have a chemical purity of at least 98% by weight, preferably have a purity of at least 99.5% by weight.

In various embodiments, the crystals of levocetirizine dihydrochloride obtained as described herein have an enantiomeric excess (ee) of at least 99%, and preferably have an ee value of at least 99.8%.

### DETAILED DESCRIPTION OF THE INVENTION

Applicants have surprisingly discovered that although precipitation of cetirizine potassium salt can be readily performed to enable its purification, the same procedure is not applicable for purifying the levocetirizine potassium salt. Therefore, an alternative approach has been discovered for purifying levocetirizine potassium salt. It has been found that levocetirizine potassium salt can be separated from its impurities using a series of extractions.

The term “enantiomeric excess” or “enantiomeric purity” (ee), as defined herein, is the percent excess of one enantiomer compared to that of the other enantiomer, and can be calculated using the following equation:

percent enantiomeric excess=((R−S)/(R+S))×100=% (R*)−% (S*)

wherein R and S are the number of moles of each enantiomer in the mixture, and R* and S* are the respective mole fractions of the enantiomers in the mixture.

The term “chemical purity,” as defined herein, refers to the liquid chromatography area percent of the peak corresponding to the levocetirizine dihydrochloride isomer relative to the area percent of the levocetirizine dihydrochloride isomer and all the other detected impurities.

Crude levocetirizine or a crude sample comprising levocetirizine, as used herein, refers to a sample having up to 88% by weight of levocetirizine. The crude sample also can contain the enantiomer of levocetirizine (i.e., dextrocetirizine).

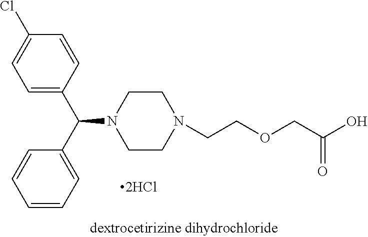

Other non-limiting examples of impurities that can be present in the crude sample include levocetirizine ethyl ester (Compound IVB), 2-[2-[4-dipheylphenymethyl-1-piperazinyl]-ethoxy]acetic acid (Compound VII), and Compound VIII.

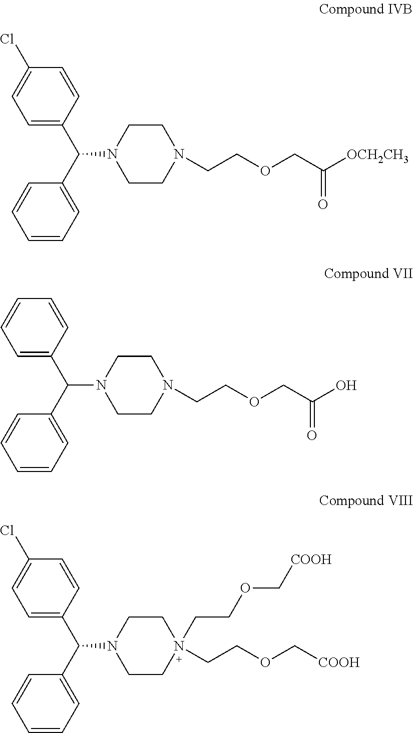

(R)-1-[(2-chlorophenyl)-phenylmethyl]-piperazine (Compound IX) was also identified as an impurity in levocetirizine, which is believed to be an impurity attributed to the starting material Compound IIB:

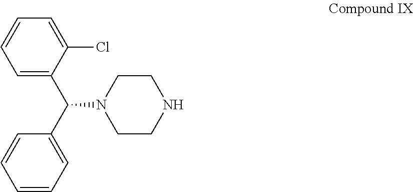

Crude levocetirizine or a salt thereof including levocetirizine potassium salt (Compound VB) and levocetirizine dihydrochloride salt can be prepared as depicted in Scheme 4 below, starting from (R)-1-[(4-chlorophenyl)-phenylmethyl]-piperazine (Compound IIB).

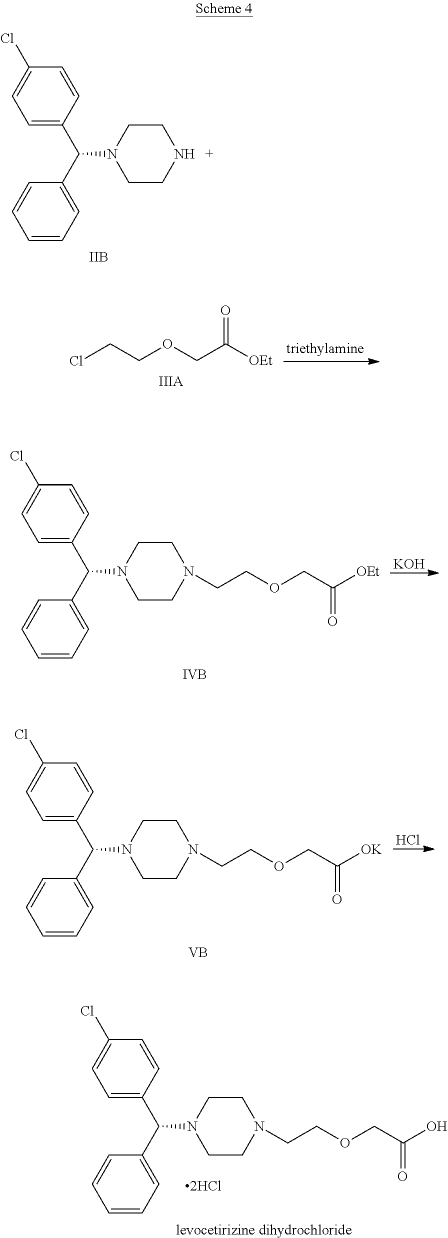

Thus, in one embodiment the present invention provides a process for preparing chemically and enantiomerically pure levocetirizine or a salt thereof, and, in particular, the dihydrochloride salt, which comprises the steps of:
a) admixing a crude sample comprising levocetirizine potassium salt with a first organic solvent and water to form a first organic phase and a first aqueous phase;
b) separating the first aqueous phase from the first organic phase;
c) adding an acid and a second organic solvent to the first aqueous phase to form a second organic phase and a second aqueous phase;
d) separating the second organic phase from the second aqueous phase;
e) distilling the second organic solvent from the second organic phase to form a residue;
f) dissolving the residue of step (e) in a third organic solvent;
g) bubbling hydrogen chloride gas through the solution of step (0 to precipitate crystals of levocetirizine dihydrochloride; and
h) optionally isolating, washing, and drying the crystals of levocetirizine dihydrochloride.

Preferably, the first organic solvent is selected from the group consisting of methyl acetate, ethyl acetate, isobutyl acetate, chloroform, and mixtures thereof. More preferably, the first organic solvent comprises ethyl acetate.

While using toluene or dichloromethane as first organic solvents instead of ethyl acetate, emulsions were obtained and the phases could not be separated.

Preferably, the second organic solvent is selected from the group consisting of dichloromethane, chloroform, toluene, diethyl ether, diisopropyl ether, methyl tert-butyl ether (MTBE), and mixtures thereof. More preferably, the second organic solvent comprises dichloromethane.

The acid is typically an inorganic acid. Preferably, the inorganic acid comprises hydrochloric acid, and more preferably, the source of hydrochloric acid is hydrogen chloride gas.

Preferably, the third organic solvent is selected from the group consisting of acetone, methyl ethyl ketone (MEK), methyl isobutyl ketone (MIBK), acetonitrile, tetrahydrofuran (THF), chloroform, methyl tert-butyl ether (MTBE), and mixtures thereof. More preferably, the third organic solvent comprises acetone.

In accordance with the present invention, purified levocetirizine dihydrochloride is obtained containing less than 0.1% of the dextrotatory isomer, preferably containing about 0.05% of the dextrotatory isomer, that is, having an ee of 99.9%.

In accordance with another embodiment of the present invention, the purified levocetirizine dihydrochloride is obtained as described herein having a chemical purity of at least 98%, preferably having a purity equal to or greater than 99.5%.

In accordance with another embodiment of the present invention, the purified crystalline levocetirizine dihydrochloride, produced as described herein, contains residual solvents of less than 500 parts per million (ppm) acetone, less than 100 ppm ethanol, and less than 50 ppm dichloromethane.

The levocetirizine dihydrochloride produced in accordance with the present invention can be used in a pharmaceutical composition, which can include levocetirizine dihydrochloride produced as described herein (e.g., in a therapeutically effective amount) and one or more pharmaceutically acceptable additives and/or excipients.

### EXAMPLES

The following examples further illustrate the invention but should not be construed as limiting its scope.

### Reference Example

This example details the preparation of (R)-2-[2-[4-[(4-chloropheyl)-phenymethyl]-1-piperazinyl]ethoxy]acetic acid potassium salt (Compound VB)

(R)-1-[(4-chlorophenyl)-phenylmethyl]-piperazine (Compound IIB), 99.8% ee (10 g, 0.035 mol), ethyl 2-chloroethoxyacetate (10 g, 0.028 mol), and triethylamine (50 ml) were introduced into a reaction vessel. The mixture was stirred at 135° C. for 14 hours, then cooled to ambient temperature and filtered. The filtrate was distilled under vacuum to remove excess ethyl 2-chloroethoxyacetate. The resulting residue was dissolved in ethanol (40 ml), and solid potassium hydroxide (5 g) was added in portions, under cooling. Then, the mixture was refluxed for 5 hours to obtain a solution of (R)-2-[2-[4-[(4-chloropheyl)phenymethyl]-1-piperazinyl]-ethoxy]acetic acid potassium salt in ethanol. A sample was withdrawn and analyzed using HPLC to reveal a purity of 87.7% of the (R)-2-[2-[4-[(4-chloropheyl)-phenymethyl]-1-piperazinyl]ethoxy]acetic acid potassium salt. The main impurities identified were levocetirizine ethyl ester (Compound VB)-8.45% by weight, (R)-1-[(2-chlorophenyl)-phenylmethyl]-piperazine (Compound IX)-2.33% by weight, and (R)-1-[(4-chlorophenyl)-phenylmethyl]-piperazine (Compound IIB)-0.82% by weight.

### EXAMPLE

This example demonstrates the preparation of levocetirizine using the purification methods disclosed herein.

A reaction vessel was charged with a solution of (R)-2-[2-[4-[(4-chloropheyl)phenymethyl]-1-piperazinyl]ethoxy]acetic acid potassium salt (87.7% purity by HPLC) in ethanol (165 ml) under mixing. The ethanol was distilled off under vacuum to afford an oily residue. Distilled water (210 ml) and ethyl acetate (250 ml) then were added and stirring was maintained for half an hour. The organic and aqueous layers were separated, and the aqueous layer, containing the potassium salt, was washed twice with ethyl acetate. A sample analyzed by HPLC revealed that the purity of the (R)-2-[2-[4-[(4-chloropheyl)-phenymethyl]-1-piperazinyl]ethoxy]acetic acid potassium salt in the aqueous phase was 97.4%, containing 0.09% of (R)-1-[(2-chlorophenyl)-phenylmethyl]-piperazine, and 0.62% of (R)-1-[(4-chlorophenyl)-phenylmethyl]-piperazine.

Hydrochloric acid (10.8 ml of a 37% solution) was added to the aqueous layer to afford a pH of 3-3.5. Dichloromethane (11.5 ml) was added, stirring was maintained for half an hour, then the phases were allowed to separate. Dichloromethane (60 ml) was added to the aqueous phase, stirring was maintained for half an hour, then the phases were allowed to separate. The organic phases from the various extractions were combined, washed with water, and the resulting organic and aqueous layers were separated. The organic solvent was distilled off, acetone (400 ml) was added to the residue, and stirring was maintained at room temperature until a clear solution was obtained. Hydrochloric gas was bubbled through the cooled clear solution until the pH of the mixture was about 1, which promoted precipitation of levocetirizine dihydrochloride salt. The resulting crystals were washed with cold acetone (20 ml), filtered, and dried to obtain levocetirizine dihydrochloride salt having 99.5% chemical purity (by HPLC). The obtained product contained less than 0.02% each of the impurities Compound VB, Compound VII, and Compound VIII. The content of the dextrotatory enantiomer was 0.05% (according to HPLC), which corresponded to an enantiomeric excess (ee) of 99.9%. The residual solvents content was less than 500 ppm of acetone, less than 50 ppm of dichloromethane, and less than 100 ppm of ethanol.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A process for purifying levocetirizine or a salt thereof, comprising:
a) admixing a crude sample comprising levocetirizine potassium salt with a first organic solvent and water to form a first organic phase and a first aqueous phase;
b) separating the first aqueous phase from the first organic phase;
c) adding an acid and a second organic solvent to the first aqueous phase to form a second organic phase and a second aqueous phase;
d) separating the second organic phase from the second aqueous phase;
e) distilling the second organic solvent from the second organic phase to form a residue;
f) dissolving the residue of step (e) in a third organic solvent;
g) bubbling hydrogen chloride gas through the solution of step (f) to precipitate crystals of levocetirizine dihydrochloride; and
h) optionally isolating, washing, and drying the crystals of levocetirizine dihydrochloride.

2. The process of claim 1, wherein the first organic solvent is selected from the group consisting of methyl acetate, ethyl acetate, isobutyl acetate, chloroform, and mixtures thereof.

3. The process of claim 2, wherein the first organic solvent comprises ethyl acetate.

4. The process of claim 1, wherein the second organic solvent is selected from the group consisting of dichloromethane, chloroform, toluene, diethyl ether, diisopropyl ether, methyl tert-butyl ether, and mixtures thereof.

5. The process of claim 4, wherein the second organic solvent comprises dichloromethane.

6. The process of claim 1, wherein the third organic solvent is selected from the group consisting of acetone, methyl ethyl ketone, methyl isobutyl ketone, acetonitrile, tetrahydrofuran, chloroform, methyl tert-butyl ether, and mixtures thereof.

7. The process of claim 6, wherein the third organic solvent comprises acetone.

8. The process of claim 1, wherein the crystals of levocetirizine dihydrochloride comprise less than 0.1% dextrocetirizine.

9. The process of claim 8, wherein the crystals of levocetirizine dihydrochloride have an enantiomeric excess of at least 99.9%.

10. The process of claim 1, wherein the crystals of levocetirizine dihydrochloride have a chemical purity of at least 98% by weight.

11. The process of claim 10, wherein the crystals of levocetirizine dihydrochloride have a chemical purity of at least 99.5% by weight.

12. The process of claim 1, wherein the crystals of levocetirizine dihydrochloride have up to 500 parts per million (ppm) acetone, up to 100 ppm ethanol, and up to 50 ppm methylene chloride.

13. A pharmaceutical composition comprising levocetirizine dihydrochloride prepared according to claim 1 and at least one pharmaceutically acceptable excipient.
